(19) United States
(12) Patent Application Publication
(10) Pub. No.: US 2004/0162558 A1
(43) Pub. Date: Aug. 19, 2004

(54) Spinal plate having an integral rod connector portion

(76) Inventors: Sajan K. Hegde, Chennai (IN); B. Thomas Barker, Bartlett, TN (US); Eric S. Heinz, Memphis, TN (US); Russell Powers, Collierville, TN (US)

Correspondence Address: Woodard, Emhardt, Naughton,, Moriarty and McNett LLP, Bank One Center/Tower, 111 Monument Circle, Suite 3700, Indianapolis, IN, 46204-5137, US

(21) Appl. No.: 10/393,073

(22) Filed: Mar. 20, 2003

Related U.S. Application Data
(60) Provisional application No. 60/448,045, filed on Feb. 18, 2003.

(51) Int. Cl. A61B 17/56

(52) U.S. Cl. 606/61

(57) ABSTRACT

A spinal plate including a plate portion having an upper side and an opposite lower side, a connector portion that is integrally attached to the upper side of the plate portion and defining a channel sized to receive an elongate rod therein, and at least one bone engagement member extending from the lower side of the plate portion and adapted for engagement with bone. In one embodiment, the connector portion is formed integral with the plate portion so as to define a unitary, single-piece structure. In another embodiment, the bone engagement member comprises one or more prongs formed integral with the plate portion and/or one or more bone screws extending through corresponding openings in the plate portion. In certain embodiments, the spinal plate includes a pair of connector portions integrally attached to the upper side of the plate portion and defining channels, each sized to receive an elongate rod therein.

### CROSS REFERENCE TO RELATED APPLICATIONS

The present application claims the benefit of Provisional Application Serial No. 60/448,045 filed on Feb. 18, 2003, the contents of which are hereby incorporated by reference in their entirety.

### FIELD OF THE INVENTION

The present invention generally relates to a device for anchoring an elongate rod to bone, and more particularly relates to a spinal plate having an integral rod connector portion.

### BACKGROUND

A variety of spinal systems and devices are known in the art for treating or correcting spinal deformities, promoting healing, or for use in other therapeutic applications. Some prior spinal systems engage an elongate bar or rod to one or more vertebrae via a number of screws or hooks to stabilize a portion of the spinal column. Among such systems are those disclosed in U.S. Pat. No. 5,005,562 to Cotrel, U.S. Pat. No. 5,797,911 to Sherman et al., and U.S. Pat. No. 6,280,442 to Barker et al. With regard to these types of systems, a U-shaped or C-shaped head extending from a screw or hook is typically used to indirectly couple an elongate rod to a number of vertebrae. Other types of spinal systems use rigid plates or staples that are engaged directly to one or more vertebrae via a number of bone screws or prongs to stabilize a portion of the spinal column. The present invention combines certain elements and feature of each of these prior systems in a novel and non-obvious manner to provide an improved device for stabilizing a portion of the spinal column.

### SUMMARY

The present invention relates generally to an improved spinal plate. While the actual nature of the invention covered herein can only be determined with reference to the claims appended hereto, certain forms of the invention that are characteristic of the preferred embodiments disclosed herein are described briefly as follows.

In one form of the invention, a spinal plate is provided, including a plate portion having a first side and an opposite second side, a connector portion integrally attached to said the side of the plate portion and defining a channel sized to receive an elongate rod therein, and at least one bone engagement member extending from the second side of said plate portion and adapted for engagement with bone.

In another form of the invention, a spinal plate is provided, including a plate portion having a first side and an opposite second side, a first connector portion integrally attached to the first side of the plate portion and defining a first channel sized to receive a first elongate rod therein, a second connector portion integrally attached to the first side of the plate portion and defining a second channel sized to receive a second elongate rod therein, and at least one bone engagement member extending from the second side of the plate portion and adapted for engagement with bone.

In another form of the invention, a spinal plate is provided, including a plate portion having a first side and an opposite second side, and defining at least one opening extending between the first and second sides, at least one connector portion formed integral with the plate portion and extending from the first side of the plate portion and defining a channel sized to receive an elongate rod therein, at least one prong formed integral with the plate portion and extending from the second side of said plate portion for engagement with bone, and at least one bone anchor having a bone engaging portion extending through the at least one opening in the plate portion from the first side to the second side for engagement with bone.

In another form of the invention, a spinal plate is provided, including a plate portion having a first side and an opposite second side, means for connecting at least one elongate rod to the plate portion, the means for connecting extending from the first side of the plate portion and formed integral therewith, and means for engaging the plate portion to bone, the means for engaging extending from the second side of the plate portion.

It is one object of the present invention to provide an improved spinal plate. Further objects, features, advantages, benefits, and further aspects of the present invention will become apparent from the drawings and description contained herein.

### BRIEF DESCRIPTION OF THE FIGURES

FIG. 1 is a top perspective view of a spinal plate according to one form of the present invention.

FIG. 2 is a side elevational view of the spinal plate illustrated in FIG. 1.

FIG. 3 is an end elevational view of the spinal plate illustrated in FIG. 1.

FIG. 4 is a top plan view of the spinal plate illustrated in FIG. 1.

FIG. 5 is a bottom view of the spinal plate illustrated in FIG. 1.

FIG. 6 is a top perspective view of a spinal plate according to another form of the present invention.

FIG. 7 is a side elevational view of the spinal plate illustrated in FIG. 6.

FIG. 8 is an end elevational view of the spinal plate illustrated in FIG. 6.

FIG. 9 is a top plan view of the spinal plate illustrated in FIG. 6.

FIG. 10 is a bottom view of the spinal plate illustrated in FIG. 6.

FIG. 11 is a lateral view of a portion of the spinal column with upper and lower ones of the spinal plate illustrated in FIG. 1 anchored to upper and lower vertebrae, respectively, with an elongate spinal rod engaged with and extending between the upper and lower spinal plates.

FIG. 12 is a superior view, partially in cross section, of the spinal construct illustrated in FIG. 11.

FIG. 13 is a lateral view of a portion of the spinal column with upper and lower ones of the spinal plate illustrated in FIG. 6 anchored to upper and lower vertebrae, respectively, with a pair of elongate spinal rods engaged with and extending between the upper and lower spinal plates.

FIG. 14 is a superior view, partially in cross section, of the spinal construct illustrated in FIG. 13.

FIG. 15 is a side perspective view of a spinal plate according to another form of the present invention.

FIG. 16 is a side perspective view of a spinal plate according to a further form of the present invention.

FIG. 17 is a side perspective view of a spinal plate according to still another form of the present invention.

FIG. 18 is a side perspective view of a spinal plate according to yet another form of the present invention.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is hereby intended, such alterations and further modifications in the illustrated devices, and such further applications of the principles of the invention as illustrated herein being contemplated as would normally occur to one skilled in the art to which the invention relates.

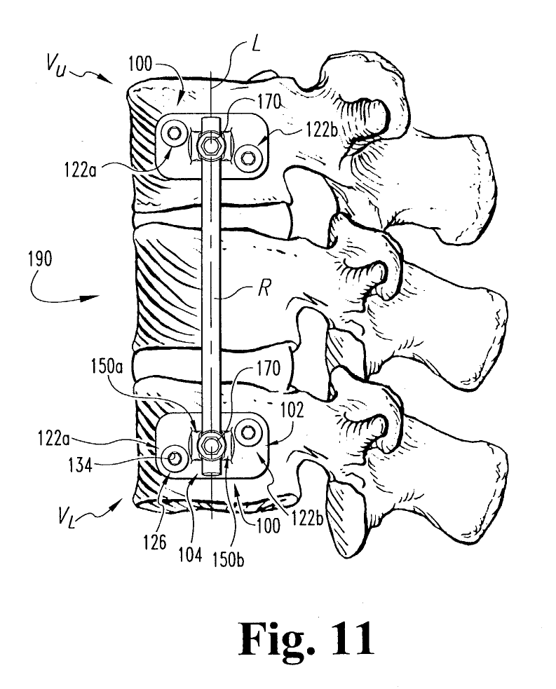

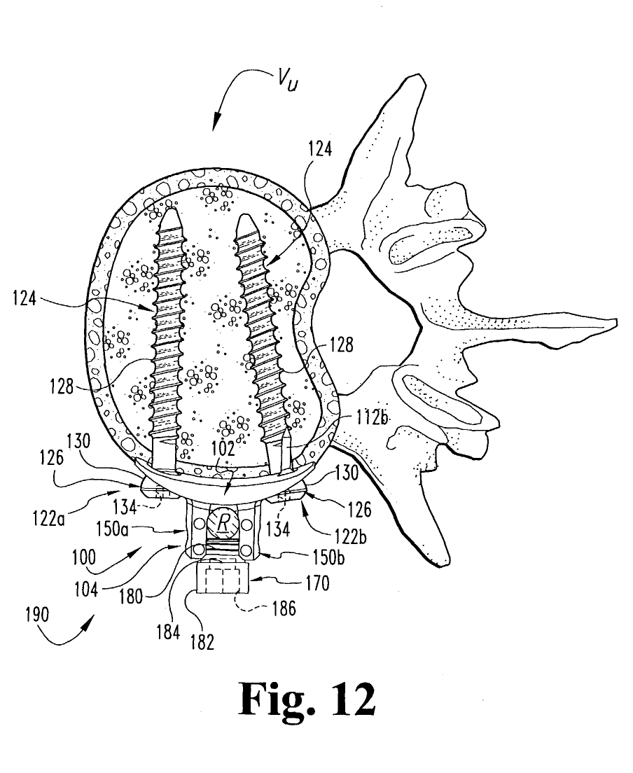

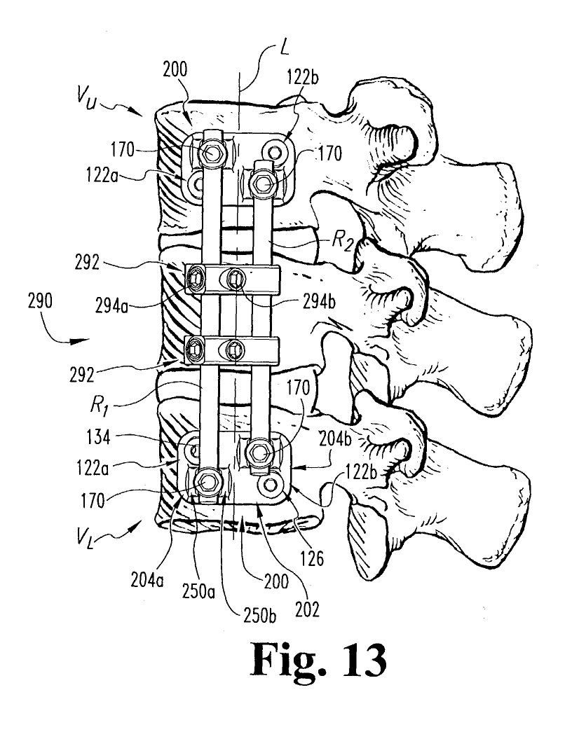

Referring to FIGS. 1-5, shown therein is a spinal plate 100 according to one form of the present invention. The spinal plate 100 is generally comprised of a plate portion 102 and a connector portion 104. The connector portion 104 is integrally attached to the plate portion 102 and defines a channel 110 sized and shaped to receive an elongate element therein, such as, for example, an elongate rod R (FIGS. 11 and 12). At least one bone engagement member extends from the plate portion 102 for engagement with bone, such as, for example, a vertebral body, to anchor the spinal plate 100 thereto. However, it should be understood that the spinal plate 100 and any other spinal plate illustrated and described herein may be engaged to other portions of the spinal column or may be used in applications outside of the spinal field.

In the illustrated embodiment of the invention, the plate portion 102 has a substantially rectangular configuration, having a length extending along a longitudinal axis L and a width extending along a transverse axis T. However, it should be understood that the plate portion 102 may take on other shapes and configurations, including non-rectangular or irregular shapes and configurations. The plate portion 102 has a first side 102a facing away from the vertebral body (hereafter referred to as the upper side) and an opposite second side 102b facing the vertebral body (hereafter referred to as the lower side).

The connector portion 104 extends from and is integrally attached to the upper side 102a of the plate portion 102. In one embodiment of the invention, the lower side 102b of the plate portion 102 defines a contour or curvature that approximates the anatomic curvature associated with the vertebral body to which the spinal plate 100 is engaged. In a specific embodiment, the lower side 102b of the plate portion 102 defines a curvature C extending generally along the transverse axis T which corresponds to the curvature of a vertebral body in an anterior-posterior or sagittal direction (FIG. 12). The lower side 102b of the plate portion 102 may also define a curvature extending generally along the longitudinal axis L which corresponds to the curvature of a vertebral body in a superior-inferior direction (not shown). In a further embodiment of the invention, the upper side 102a of the plate portion 102 is also curved and is substantially smooth to minimize irritation or trauma to adjacent soft tissue or vascular structures.

In one embodiment of the invention, a pair of prong members or spikes 112a, 112b extends from the lower side 102b of the plate portion 102 to engage the spinal plate 100 to bone. The prongs 112a, 112b are preferably formed integral with the plate portion 102. However, it should be understood that other means and methods for attaching the prongs 112a, 122b to the plate portion 102 are also contemplated as falling within the scope of the present invention, such as, for example, by welding, bonding, fastening, or any other method of attachment known to those of skill in the art. The prongs 112a, 112b each preferably define a pointed tip 114 to facilitate insertion into bone. Although the illustrated embodiment of the spinal plate 100 includes a pair of prongs 112a, 112b, it should be understood that the spinal plate 100 may include any number of prongs, including a single prong or three or more prongs. It should also be understood that in other embodiments of the invention, the spinal plate 100 need not include any prongs, relying instead upon other types and configurations of bone engagement members to anchor the spinal plate 100 to bone, such as, for example, the bone anchors 122a, 122b illustrated in FIGS. 11 and 12 and discussed below.

As illustrated in FIG. 5, the plate portion 102 defines an outer peripheral edge 116 extending about an interior region 118, with the prongs 112a, 112b extending from the interior region 118 of the plate portion 102 in an inboard manner relative to the outer peripheral edge 116. In one embodiment of the invention, the prongs 112a, 112b are arranged diagonally opposite one another relative to the longitudinal axis L and the transverse axis T, positioned at opposite corners of the plate portion 102. However, it should be understood that other arrangements and positions of the prongs 112a, 112b are also contemplated as falling within the scope of the present invention.

In one embodiment of the invention, the plate portion 102 defines a pair of openings 120a, 120b extending from the upper side 102a to the lower side 102b. The openings 120a, 120b are sized and shaped to receive a respective bone anchor member 122a, 122b therethrough (FIGS. 11 and 12) to engage the spinal plate 100 to bone. Although the illustrated embodiment of the spinal plate 100 includes a pair of bone anchor openings 120a, 120b configured to receive a corresponding pair of bone anchors 122a, 122b, it should be understood that the spinal plate 100 may define any number of openings for receiving a corresponding number of bone anchors, including a single opening or three or more openings. It should also be understood that in other embodiments of the invention, the spinal plate 100 need not define any openings for receiving bone anchors, relying instead upon other types and configurations of bone engagement members to anchor the spinal plate 100 to bone, such as, for example, the prongs 112a, 112b illustrated and described above.

As shown in FIG. 12, in one embodiment of the invention, the bone anchors 122a, 122b are configured as bone screws. However, it should be understood that other types and configurations of bone anchors or bone engagement members are also contemplated, such as, for example, pins, nails, staples, U-shaped or L-shaped hook elements adapted for engagement with the pedicle or spinous process of a vertebra, or any other element or member adapted for engagement with bone that would occur to one of skill in the art. Each of the bone screws 122a, 122b includes a shank portion 124 and a head portion 126. The shank portion 124 defines threads 128 that are configured to engage bone, such as, for example, cancellous bone. The threaded shank portion 124 of the bone screws 122a, 122b is sized and shaped to pass through the respective bone anchor openings 120a, 120b in the plate portion 102 from the upper side 102a to the lower side 102b. The head portion 126 is sized and shaped to prevent passage through the respective bone anchor openings 120a, 120b in the plate portion 102 adjacent the upper side 102a to thereby engage the plate portion 102 to bone. In one embodiment of the invention, the head portion 126 includes an arcuate-shaped lower portion 130 configured for engagement within an arcuate-shaped recess 132 formed about each of the bone anchor openings 120a, 120b adjacent the upper side 102a of the plate portion 102. In a specific embodiment, the arcuate-shaped lower portion 130 and the arcuate-shaped recess 132 each have a spherical configuration to allow the bone screws 122a, 122b to be oriented within a range of angles relative to the plate portion 102 and relative to one another. In a further embodiment of the invention, the head portion 126 defines a tool receiving recess 134 sized and shaped to receive the distal end portion of a driving tool, such as, for example, a screw driver (not shown) to aid in driving the bone screws 122a, 122b into bone.

As shown in FIG. 4, in one embodiment of the invention, the bone anchor openings 120a, 120b are axially and transversely offset from the connector portion 104. However, as will be illustrated and described below, at least one of the bone anchor openings may be generally aligned with the connector portion 104. As also shown in FIG. 4, the bone anchor openings 120a, 120b are arranged diagonally opposite one another relative to the longitudinal axis L and the transverse axis T, positioned at opposite corners of the plate portion 102. As illustrated in FIG. 5, the openings 120a, 120b are arranged diagonally opposite the prongs 112a, 12b. However, it should be understood that other arrangements and positions of the bone anchor openings 120a, 120b are also contemplated as falling within the scope of the present invention.

As illustrated and described above, the connector portion 104 is integrally attached to the plate portion 102 and defines a channel 110 sized and shaped to receive an elongate rod R therein. In one embodiment of the invention, the connector portion 104 is oriented such that the channel 110 is generally aligned along the longitudinal axis L. However, other orientations of the connector portion 104 are also contemplated as falling with the scope of the present invention, including orientations where the channel 110 is aligned generally along the transverse axis T or along another axis oriented at an oblique angle relative to the longitudinal axis L or the transverse axis T.

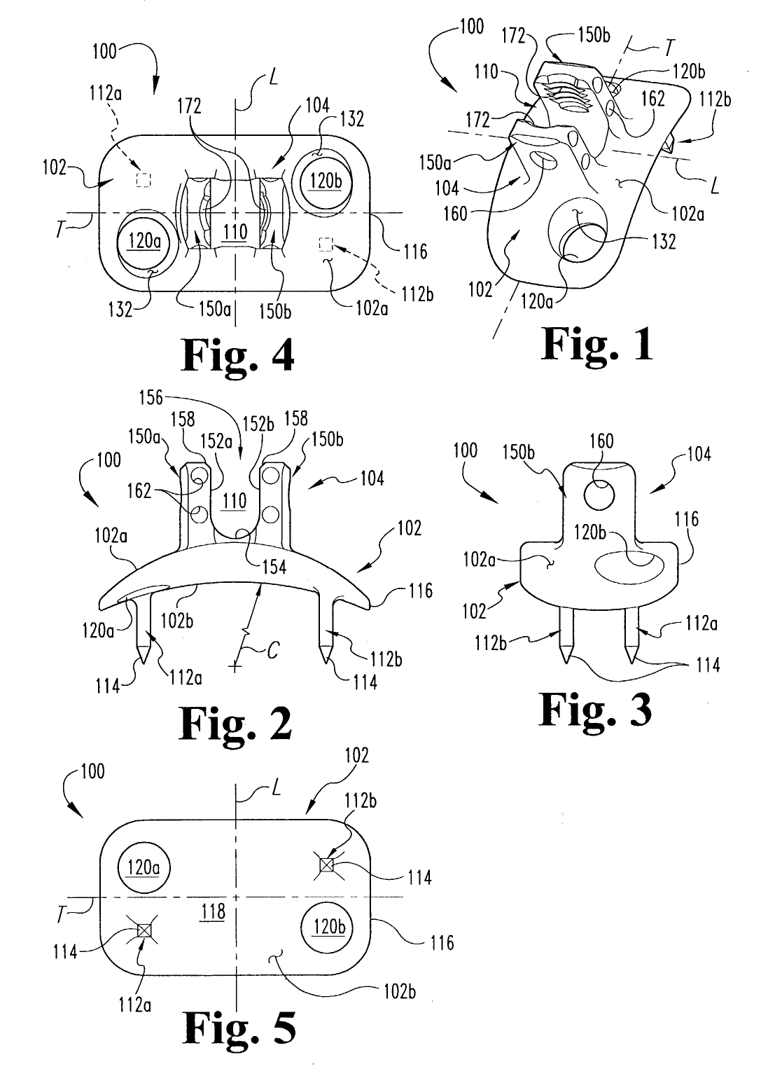

The connector portion 104 is preferably formed integral with the plate portion 102 so as to define a unitary, single-piece structure. However, it should be understood that other means and methods for attaching the connector portion 104 to the plate portion 102 are also contemplated as falling within the scope of the present invention, such as, for example, by welding, bonding, fastening, or any other method of attachment known to those of skill in the art. As illustrated in FIG. 4, the connector portion 104 is disposed intermediate the bone anchor openings 120a, 120b in both an axial direction along the longitudinal axis L and a transverse direction along the transverse axis T. As illustrated in FIGS. 2 and 3, the connector portion 104 is also disposed intermediate the prongs 112a, 112b in both an axial direction and a transverse direction. However, it should be understood that other arrangements and positions of the connector portion 104 are also contemplated as falling within the scope of the present invention.

In one embodiment of the invention, the connector portion 104 includes a pair of legs or posts 150a, 150b extending from the upper side 102a of the plate portion 102 and spaced apart to define the channel 110 therebetween. In the illustrated embodiment of the spinal plate 100, the legs 150a, 150b are arranged substantially parallel to one another; however, other arrangements and orientations of the legs 150a, 150b are also contemplated. In one embodiment of the invention, the channel 110 defined by the connector portion 104 is substantially U-shaped, bound by opposing side walls 152a, 152b defined by the legs 150a, 150b, respectively, and a bottom wall 154. The side walls 152a, 152b are spaced apart a distance sufficient to receive the elongate rod R therebetween. In one embodiment of the invention, the elongate rod R has a substantially cylindrical configuration and the bottom wall 154 has a corresponding arcuate or circular configuration. However, it should be understood that the elongate rod R and/or the bottom wall 154 may define other shapes and configurations, such as, for example, an elliptical configuration, a rectangular configuration, or a polygonal configuration.

In the illustrated embodiment of the invention, the connector portion 104 defines an upper opening 156 between the distal end portions of the legs 150a, 150b and communicating with the channel 110. The upper opening 156 is sized to receive the elongate rod R therethrough for passage into the channel 110. The side walls 152a, 152b adjacent the distal end portions of the legs 150a, 150b may define inwardly tapering portions 158 to facilitate insertion of the elongate rod R through the upper opening 156 and into the channel 110. The outer surfaces of the legs 150a, 150b may also define a recess 160 and the end surfaces of the legs 150a, 150b may define a number of recesses 162 that are adapted to receive an end portion of an insertion instrument or driving tool (not shown) therein to aid in manipulating or driving the spinal plate 100. In one embodiment, the recesses 160 and 162 have a substantially circular configuration and may define a slight inward taper to facilitate insertion of a corresponding portion of an instrument or tool therein. The recesses 160, 162 may also be threaded to provide threading engagement with an instrument or tool.

In one embodiment of the invention, the elongate rod R is receivable within the channel 110 in a direction transverse to the longitudinal axis L. In a more specific embodiment of the invention, the elongate rod R is receivable within the channel 110 in a direction substantially perpendicular to the longitudinal axis L. Although the connection portion 104 has been illustrated and described as a top loading-type device configured to receive the elongate rod R through the upper opening 156 and into the channel 110, it should be understood that the connection portion 104 may alternatively be configured as a side loading-type device wherein the elongate rod R is inserted through a lateral opening in the connection portion 104 and into the channel 110. It should also be understood that the elongate rod R may alternatively be inserted into the channel 110 in an axial direction along the longitudinal axis L, thereby eliminating the need for the upper opening 156 between the distal ends of the legs 150a, 150b or a lateral opening communicating with the channel 110.

In a further embodiment of the invention, a clamp member 170 (FIGS. 11 and 12) is provided to clamp or capture the elongate rod R within the channel 110. In one embodiment, the clamp member 170 is displaceable along the legs 150a, 150b of the connector portion 104 and into engagement with the elongate rod R. In a specific embodiment of the invention, the clamp member 170 is configured as a setscrew adapted to threadingly engage internal threads 172 (FIGS. 1 and 4) formed along at least a portion of the inner walls 152a, 152b of the legs 150a, 150b. However, it should be understood that other types and configurations of clamp members are also contemplated as falling within the scope of the present invention. For example, the elongate rod R may be captured or clamped within the channel 110 by a ring or nut engagable about the outer surfaces of the legs 150a, 150b, or by a cam lock member, a sliding member, or any other type of member that would occur to one of skill in the art for capturing the elongate rod R within the channel 110.

As illustrated in FIG. 12, the setscrew 170 includes a threaded portion 180 adapted for engagement with the internal threads 172 defined by the connector portion 104, and a head portion 182 extending from the threaded portion 180 and adapted for engagement by a driving tool. In one embodiment of the invention, the head portion 182 is selectively removable from the threaded portion 180. In a specific embodiment, the head portion 182 is attached to the threaded portion 180 by a frangible region or fracture initiator 184 adapted to allow the head portion 182 to be snapped off or broken away from the threaded portion 180 once the threaded portion 180 is engaged against the elongate rod R. The head portion 182 preferably defines a recess 186 configured to receive a distal end portion of a driving tool therein, such as, for example, the distal end portion of a screwdriver.

Referring to FIGS. 11 and 12, shown therein is a spinal construct 190 comprising a pair of the spinal plates 100 engaged to upper and lower vertebrae V_{U}, V_{L}, respectively, and an elongate rod R extending between the spinal plates 100. As should be appreciated, the spinal construct 190 functions to fixate or stabilize the portion of the spinal column between the upper and lower vertebrae V_{U}, V_{L}. In one embodiment of the invention, the spinal construct 190 is used to fixate or stabilize a portion of the thoracic or thoracolumbar region of the spine. However, it should be understood that the spinal construct 190 may be used in association with other regions of the spine, including the cervical or lumbar regions of the spine. In the illustrated embodiment of the invention, the spinal plates 100 are laterally engaged to anterior portions of the upper and lower vertebrae V_{U}, V_{L}. However, it should be understood that the spinal plates 100 may alternatively be engaged to other portions of the upper and lower vertebrae V_{U}, V_{L} A pair of spinal plates 100 is initially engaged to anterior portions of the upper and lower vertebrae V_{U}, V_{L}, respectively, via driving the prongs 112a, 112b into vertebral bone. As shown in FIG. 1, the upper and lower spinal plates 100 are preferably oriented such that the channels 110 defined by the connector portions 104 are substantially aligned with one another generally along the longitudinal axis L.

However, it should be understood that the upper and lower spinal plates 100 may be oriented such that the channels 110 are angularly offset relative to one other. In one embodiment of the invention, an impactor-type instrument, such as, for example, a staple impactor, may be used to drive the prongs 112a, 112b of the spinal plates 100 into bone. Pilot holes may then be preformed in the upper and lower vertebrae V_{U}, V_{L} via a drill or awl instrument for receiving the bone screws 122a, 122b. However, it should be understood that the bone screws 122a, 122b may alternatively be configured with self-drilling/self-cutting features to eliminate the need for forming pilot holes. A drill/awl guide and/or a screw guide may be used to insure that the pilot holes and bone screws 122a, 122b are oriented at the appropriate angle or screw trajectory. In the illustrated embodiment of the invention, the anterior bone screw 122a is substantially aligned with the coronal plane, while the posterior bone screw 122b is oriented approximately 10 degrees in an anterior direction relative to the coronal plane. However, it should be understood that other angular orientations of the bone screws 122a, 122b are also contemplated as falling within the scope of the present invention.

Following the initial engagement of the spinal plates 100 to the upper and lower vertebrae V_{U}, V_{L} via insertion of the prongs 112a, 112b into vertebral bone, the bone screws 122a, 122b are inserted through the bone anchor openings 120a, 120b in the spinal plates 100 and the threaded shank portions 124 are driven into vertebral bone via a driving instrument, such as, for example, a screwdriver. The bone screw 122a, 122b are driven into the vertebral bone until the lower spherical portions 130 of the screw heads 126 are tightly engaged within the spherical shaped recesses 132 surrounding the bone anchor openings 120a, 120b to provide further engagement of the spinal plates 100 to the upper and lower vertebrae V_{U}, V_{L}. As should be appreciated, engagement between the lower spherical portions 130 of the screw heads 126 and the spherical shaped recesses 132 in the plate portion 102 allows the bone screws 120a, 120b to be oriented within a range of angles relative to the spinal plate 100.

Following engagement of the spinal plates 100 to the upper and lower vertebrae V_{U}, V_{L}, a distractor instrument or vertebral body spreader may be used to distract the upper and lower vertebrae V_{U}, V_{L} to a desired reduction. In one embodiment, end portions of the distractor instrument may be engaged with the connector portions 104, and more particularly within the channels 110 in the connector portions 104, to transmit the distraction or reduction forces through the spinal plates 100 to the upper and lower vertebrae V_{U}, V_{L}. The elongate rod R may then be inserted through the upper openings 156 between the legs 150a, 150b of the connector portions 104 and into the channels 110.

As discussed above, the elongate rod R is receivable within the channels 110 in the connector portions 104 in a direction substantially perpendicular to the longitudinal axis L (e.g., in a top-loading manner). However, in other embodiments of the invention, the connector portions 104 may be configured to receive the elongate rod R within the channels 110 in a lateral direction (e.g., in a side-loading manner) or in an axial direction (e.g., in the direction of the longitudinal axis L). As would be appreciated by those of skill in the art, the elongate rod R may be bent or contoured to a particular curvature either prior to or subsequent to engagement with the connector portions 104 of the upper and lower spinal plates 100.

After the elongate rod R is inserted within the channels 110 in the spinal plates 100, a clamp member or setscrew 170 is engaged against the elongate rod R to capture or clamp the elongate rod R within the channels 110 to thereby secure the elongate rod R to the upper and lower spinal plates 100. As discussed above, the setscrews 170 are threadingly advanced along the internal threads 172 formed along the interior surfaces 152a, 152b of the legs 150a, 150b until the distal ends of the setscrews 170 are tightly engaged against the outer surface of the elongate rod R. The distal ends of the setscrews 170 may be provided with pointed tips, teeth or a roughened surface for more secure engagement with the elongate rod R to further inhibit displacement or rotation of the rod R within the channels 110. As illustrated in FIG. 12, the upper head portions 182 of the setscrews 170 may be snapped off or broken away from the threaded portions 180 engaged between the legs 150a, 150b of the connector portion 104 to provide the spinal plates 100 and the spinal construct 190 with a lower overall profile.

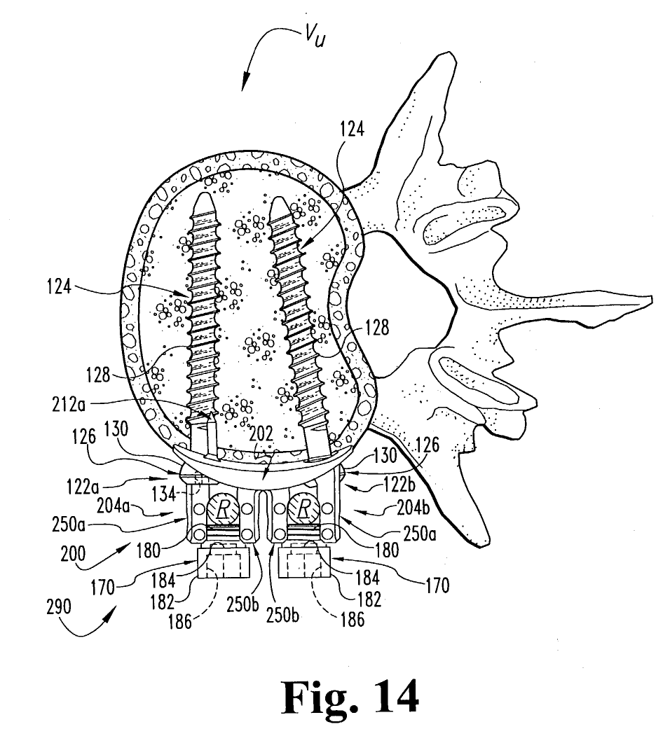

Referring to FIGS. 6-10, shown therein is a spinal plate 200 according to another form of the present invention. The spinal plate 200 is configured similar to the spinal plate 100 illustrated and described above, including a plate portion 202 configured substantially similar to the plate portion 102. However, unlike the spinal plate 100, which includes a single connector portion 104, the spinal plate 200 includes a pair of connector portions 204a, 204b integrally attached to the plate portion 202. The connector portions 204a, 204b define a pair of channels 210a, 210b, respectively, that are sized and shaped to receive a corresponding pair of elongate elements therein, such as, for example, a pair of elongate rods R₁ and R₂ (FIGS. 13 and 14). At least one bone engagement member extends from the plate portion 202 for engagement with bone, such as, for example, a vertebral body, to anchor the spinal plate 200 thereto.

In the illustrated embodiment of the invention, the plate portion 202 has a substantially rectangular configuration, having a length extending along a longitudinal axis L and a width extending along a transverse axis T. However, it should be understood that the plate portion 202 may take on other shapes and configurations, including non-rectangular or irregular shapes and configurations. The plate portion 202 has a first side 202a facing away from the vertebral body (hereafter referred to as the upper side) and an opposite second side 202b facing the vertebral body (hereafter referred to as the lower side).

The connector portions 204a, 204b extend from and are integrally attached to the upper side 202a of the plate portion 202. In one embodiment of the invention, the lower side 202b of the plate portion 202 defines a contour or curvature that approximates the anatomic curvature associated with the vertebral body to which the spinal plate 200 is engaged. Similar to the plate portion 102 of the spinal plate 100, the lower side 202b of the plate portion 202 defines a curvature C extending generally along the transverse axis T which corresponds to the curvature of a vertebral body in an anterior-posterior or sagittal direction (FIG. 14), and may also define a curvature extending generally along the longitudinal axis L which corresponds to the curvature of a vertebral body in a superior-inferior direction (not shown).

In one embodiment of the invention, a pair of prong members or spikes 212a, 212b extends from the lower side 202b of the plate portion 202 to engage the spinal plate 200 to bone. The prongs 212a, 212b are preferably formed integral with the plate portion 202. However, it should be understood that other means and methods for attaching the prongs 212a, 212b to the plate portion 202 are also contemplated as falling within the scope of the present invention, such as, for example, by welding, bonding, fastening, or any other method of attachment known to those of skill in the art. The prongs 212a, 212b each preferably define a pointed tip 214 to facilitate insertion into bone. Although the illustrated embodiment of the spinal plate 200 includes a pair of prongs 212a, 212b, it should be understood that the spinal plate 200 may include any number of prongs, including a single prong or three or more prongs. It should also be understood that in other embodiments of the invention, the spinal plate 200 need not include any prongs, relying instead upon other types and configurations of bone engagement members to anchor the spinal plate 200 to bone, such as, for example, the bone anchors 122a, 122b illustrated in FIGS. 13 and 14 and discussed below.

As illustrated in FIG. 10, the plate portion 202 defines an outer peripheral edge 216 extending about an interior region 218, with the prongs 212a, 212b extending from the interior region 218 of the plate portion 202 in an inboard manner relative to the outer peripheral edge 216. In one embodiment of the invention, the prongs 212a, 212b are arranged diagonally opposite one another relative to the longitudinal axis L and the transverse axis T, positioned at opposite corners of the plate portion 202. However, it should be understood that other arrangements and positions of the prongs 212a, 212b are also contemplated as falling within the scope of the present invention.

In one embodiment of the invention, the plate portion 202 defines a pair of openings 220a, 220b extending from the upper side 202a to the lower side 202b. The openings 220a, 220b are sized and shaped to receive a respective bone anchor member 122a, 122b therethrough (FIGS. 13 and 14) to engage the spinal plate 200 to bone. Although the illustrated embodiment of the spinal plate 200 includes a pair of bone anchor openings 220a, 220b configured to receive a corresponding pair of bone anchors 122a, 122b, it should be understood that the spinal plate 200 may define any number of bone anchor openings, including a single opening or three or more openings. It should also be understood that in other embodiments of the invention, the spinal plate 200 need not define any openings for receiving bone anchors, relying instead upon other types and configurations of bone engagement members to anchor the spinal plate 200 to bone, such as, for example, the prongs 212a, 212b illustrated and described above.

As shown in FIG. 14, in one embodiment of the invention, the bone anchors 122a, 122b are configured as bone screws including a threaded shank portion 124 and a head portion 126. The threaded shank portion 124 of the bone screws 122a, 122b is sized and shaped to pass through the respective bone anchor openings 220a, 220b in the plate portion 202 from the upper side 202a to the lower side 202b. The head portion 126 is sized and shaped to prevent passage through the respective bone anchor openings 220a, 220b in the plate portion 202 adjacent the upper side 202a to thereby engage the plate portion 202 to bone. In a specific embodiment, the arcuate-shaped lower portion 130 of the bone screws 122a, 122b is engaged with an arcuate-shaped recess 232 surrounding each of the bone anchor openings 220a, 220b to allow the bone screws 122a, 122b to be oriented within a range of angles relative to the plate portion 202 and relative to one another.

As shown in FIG. 9, in one embodiment of the invention, the bone anchor openings 220a, 220b are axially and transversely offset from the connector portions 204a, 204b, and are arranged diagonally opposite one another relative to the longitudinal axis L and the transverse axis T, positioned at opposite corners of the plate portion 202. As illustrated in FIG. 10, the openings 220a, 220b are arranged diagonally opposite the prongs 212a, 212b. However, it should be understood that other arrangements and positions of the bone anchor openings 220a, 220b relative to the connector portions 204a, 204b, the prongs 212a, 212b, and to each other are also contemplated as falling within the scope of the present invention.

As illustrated and described above, the connector portions 204a, 204b are integrally attached to the plate portion 202 and define a pair of channels 210a, 210b, respectively, that are sized and shaped to receive a pair of elongate rods R₁, R₂ therein. In one embodiment of the invention, the connector portions 204a, 204b are oriented such that each of the channels 210a, 210b are generally aligned along the longitudinal axis L and arranged substantially parallel to one another. However, it should be understood that other orientations of the connector portions 204a, 204b are also contemplated as falling with the scope of the present invention, including orientations where the channels 210a, 210b are aligned generally along the transverse axis T or along another axis oriented at an oblique angle relative to the longitudinal axis L or the transverse axis T. It should also be understood that the connector portions 204a, 204b need not necessarily be aligned along a common axis, but may instead be arranged such that the channels 210a, 210b are aligned along axes that are angularly offset from one another.

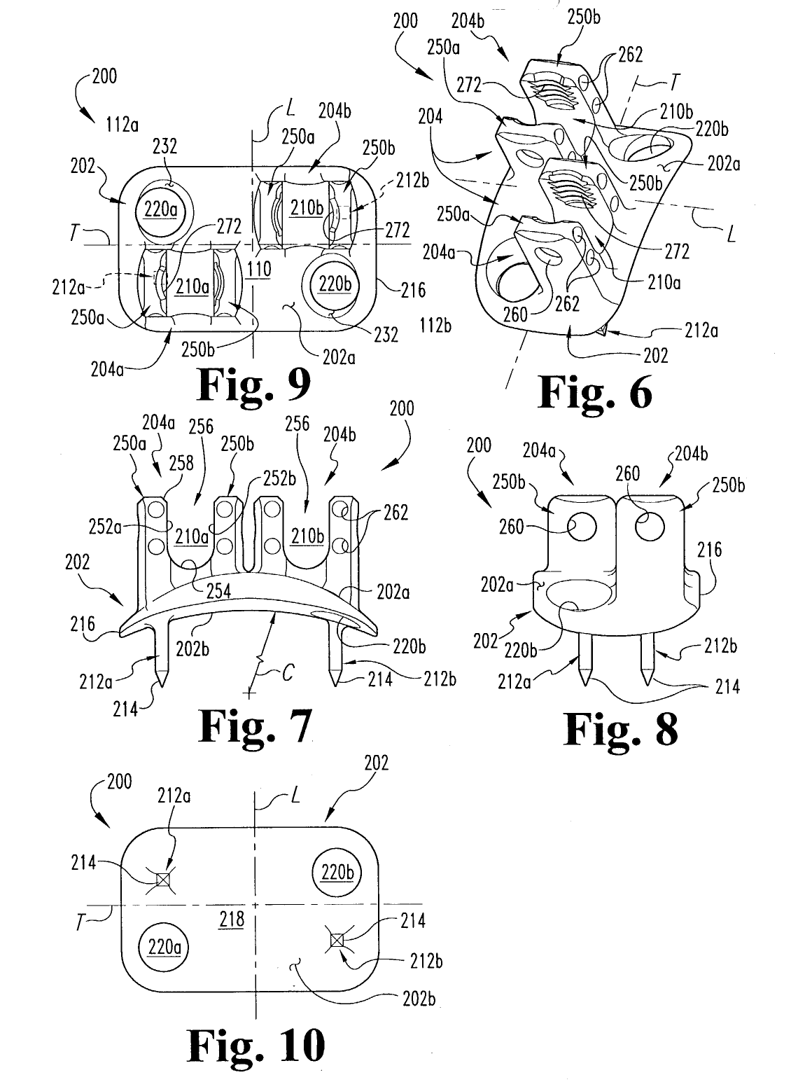

The connector portions 204a, 204b are preferably formed integral with the plate portion 202 so as to define a unitary, single-piece structure. However, it should be understood that other means and methods for attaching the connector portions 204a, 204b to the plate portion 202 are also contemplated as falling within the scope of the present invention, such as, for example, by welding, bonding, fastening, or any other method of attachment known to those of skill in the art. In one embodiment of the invention, the connector portions 204a, 204b are arranged diagonally opposite one another relative to the longitudinal axis L and the transverse axis T, positioned at opposite corners of the plate portion 202. As illustrated in FIGS. 7-9, the connector portion 204a is generally aligned with the bone anchor opening 220a in an axial direction and with the bone anchor 220b in a transverse direction, and extends generally opposite the prong 212a. Similarly, the connector portion 204b is generally aligned with the bone anchor opening 220b in an axial direction and with the bone anchor 220a in a transverse direction, and extends generally opposite the prong 212b. This particular arrangement of the connector portions 204a, 204b, the prongs 212a, 212b, and the bone anchor openings 220a, 220b securely engages the spinal plate 200 to the vertebral body with a relatively high degree of stability. However, it should be understood that other arrangements and positions of the connector portions 204a, 204b are also contemplated as falling within the scope of the present invention.

In one embodiment of the invention, the connector portions 204a, 204b each include a pair of legs or posts 250a, 250b extending from the upper side 202a of the plate portion 202 and spaced apart to define the channels 210a, 210b therebetween. In the illustrated embodiment of the spinal plate 200, the legs 250a, 250b are arranged substantially parallel to one another; however, other arrangements and orientations of the legs 250a, 250b are also contemplated. In one embodiment of the invention, the channels 210a, 210b are substantially U-shaped, bound by opposing side walls 252a, 252b defined by the legs 250a, 250b, respectively, and a bottom wall 254. The side walls 252a, 252b are spaced apart a distance sufficient to receive a respective one of the elongate rods R₁, R₂ therebetween. In one embodiment of the invention, the elongate rods R₁, R₂ have a substantially cylindrical configuration and the bottom wall 254 has a corresponding arcuate or circular configuration. However, it should be understood that the elongate rods R₁, R₂ and the bottom wall 254 may define other shapes and configurations, such as, for example, an elliptical configuration, a rectangular configuration, or a polygonal configuration.

In the illustrated embodiment of the invention, the connector portions 204a, 204b each define an upper opening 256 between the distal end portions of the legs 250a, 250b and communicating with the channels 210a, 210b. The upper opening 256 is sized to receive a respective one of the elongate rods R₁, R₂ therethrough for passage into the respective channel 210a, 210b. The side walls 252a, 252b adjacent the distal end portions of the legs 250a, 250b may define inwardly tapering portions 258 to facilitate insertion of the elongate rods R₁, R₂ through the upper openings 256 and into the channels 210a, 210b. The outer surfaces of the legs 250a, 250b may also define a recess 260 and the end surfaces of the legs 250a, 250b may define a number of recesses 262 that are adapted to receive an end portion of an insertion instrument or driving tool (not shown) therein to aid in manipulating or driving the spinal plate 200. Similar to the connector portion 104 of the spinal plate 100, the connector portions 204a, 204b may be configured to receive the elongate rods R₁, R₂ within the channels 210a, 210b in a top or side-loading manner, or the elongate rods R₁, R₂ may be inserted into the channels 210a, 210b in an axial direction.

Referring to FIGS. 13 and 14, shown therein is a spinal construct 290 comprising a pair of the spinal plates 200 engaged to upper and lower vertebrae Vu, V_{L}, respectively, and a pair of elongate rods R₁, R₂ extending between the upper and lower spinal plates 200. Similar to the spinal construct 190 illustrated and described above, the spinal construct 290 functions to fixate or stabilize the portion of the spinal column between the upper and lower vertebrae V_{U}, V_{L}. In one embodiment of the invention, the spinal construct 290 is used to fixate or stabilize a portion of the thoracic or thoracolumbar region of the spine. However, it should be understood that the spinal construct 290 may be used in association with other regions of the spine, including the cervical or lumbar regions of the spine. In the illustrated embodiment of the invention, the spinal plates 200 are laterally engaged to anterior portions of the upper and lower vertebrae V_{U}, V_{L}. However, it should be understood that the spinal plates 200 may alternatively be engaged to other portions of the upper and lower vertebrae V_{U}, V_{L}.

The spinal plates 200 are initially engaged to anterior portions of the upper and lower vertebrae V_{U}, V_{L}, respectively, via driving the prongs 212a, 212b into vertebral bone. As shown in FIG. 14, the upper and lower spinal plates 200 are preferably oriented such that the channels 210a, 210b defined by the connector portions 204a, 204b are substantially aligned with one another generally along the longitudinal axis L. However, it should be understood that the upper and lower spinal plates 200 may be oriented such that the channels 210a, 210b of the upper and lower spinal plates 200 are angularly offset relative to one other. Pilot holes may be formed in the upper and lower vertebrae V_{U}, V_{L} via a drill or awl instrument for receiving the bone screws 122a, 122b. In the illustrated embodiment of the invention, the anterior bone screw 122a is substantially aligned with the coronal plane, while the posterior bone screw 122b is oriented approximately 10 degrees in an anterior direction relative to the coronal plane. However, it should be understood that other angular orientations of the bone screws 122a, 122b are also contemplated as falling within the scope of the present invention. Following the initial engagement of the spinal plates 200 to the upper and lower vertebrae V_{U}, V_{L} via insertion of the prongs 212a, 212b into vertebral bone, the bone screws 122a, 122b are inserted through the bone anchor openings 220a, 220b in the spinal plates 200 and the threaded shank portions 124 are driven into vertebral bone by a driving instrument, such as, for example, a screwdriver. The bone screw 122a, 122b are driven into the vertebral bone until the lower spherical portions 130 of the screw heads 126 are tightly engaged within the spherical shaped recesses 232 surrounding the bone anchor openings 220a, 220b to provide further engagement of the spinal plates 200 to the upper and lower vertebrae V_{U}, V_{L}. As should be appreciated, engagement between the lower spherical portions 130 of the screw heads 126 and the spherical shaped recesses 232 in the plate portion 202 allows the bone screws 120a, 120b to be oriented within a range of angles relative to the spinal plate 200.

Following engagement of the spinal plates 200 to the upper and lower vertebrae V_{U}, V_{L}, a distractor instrument or vertebral body spreader may be used to distract the upper and lower vertebrae V_{U}, V_{L} to a desired reduction. The elongate rods R₁, R₂ are then inserted into the channels 210a, 210b in the connector portions 204a, 204b of the plates 200 in a direction substantially perpendicular to the longitudinal axis L (e.g., in a top-loading manner). However, as discussed above, the connector portions 204a, 204b may be configured to receive the elongate rods R₁, R₂ within the channels 210a, 210b in a lateral direction (e.g., in a side-loading manner) or in an axial direction (e.g., along the longitudinal axis L).

After the elongate rods R₁, R₂ are inserted within the channels 210a, 210b, setscrews 170 are threaded along the internal threads 272 of the connector portions 204a, 204b and into engagement with the elongate rods R₁, R₂ to capture or clamp the elongate rods R₁, R₂ within the channels 210a, 210b and secure the elongate rods R₁, R₂ to the upper and lower spinal plates 200. The upper head portions 182 of the setscrews 170 may be snapped off or broken away from the threaded portions 180 engaged between the legs 250a, 250b of the connector portions 204a, 204b to provide the spinal plates 200 and the spinal construct 290 with a lower overall profile.

Referring to FIG. 13, the elongate rods R₁, R₂ may be linked or coupled together by one or more cross connectors 292 to provide an added degree of structural rigidity and support to the spinal construct 290. Although a pair of the cross connectors 292 have been illustrated and described for use in association with the spinal construct 290, it should be understood that any number of connector members 290 may be used. Each of the cross connectors 292 includes a pair of channels (not shown) that are configured to receive respective ones of the elongate rods R₁, R₂ therein, and a pair of clamp members or setscrews 294a, 294b for capturing or clamping the elongate rods R₁, R₂ within the channels of the cross connector 292.

In the illustrated embodiment of the invention, the channels 210a, 210b are configured to accommodate elongate rods R₁, R₂ of substantially the same size and shape. However, it should be understood that the connector portions 204a, 204b may be configured differently so as to accommodate elongate rods having different sizes and/or configurations. In one embodiment of the invention, the spinal plates 200 are configured and arranged such that the anterior elongate rod R₁ is somewhat longer than the posterior elongate rod R₂. However, in other embodiments of the invention, the spinal plates 200 may be configured and arranged such that the anterior elongate rod R₁ is shorter than the posterior elongate rod R₂, or the anterior and posterior rods R₁, R₂ are approximately the same length.

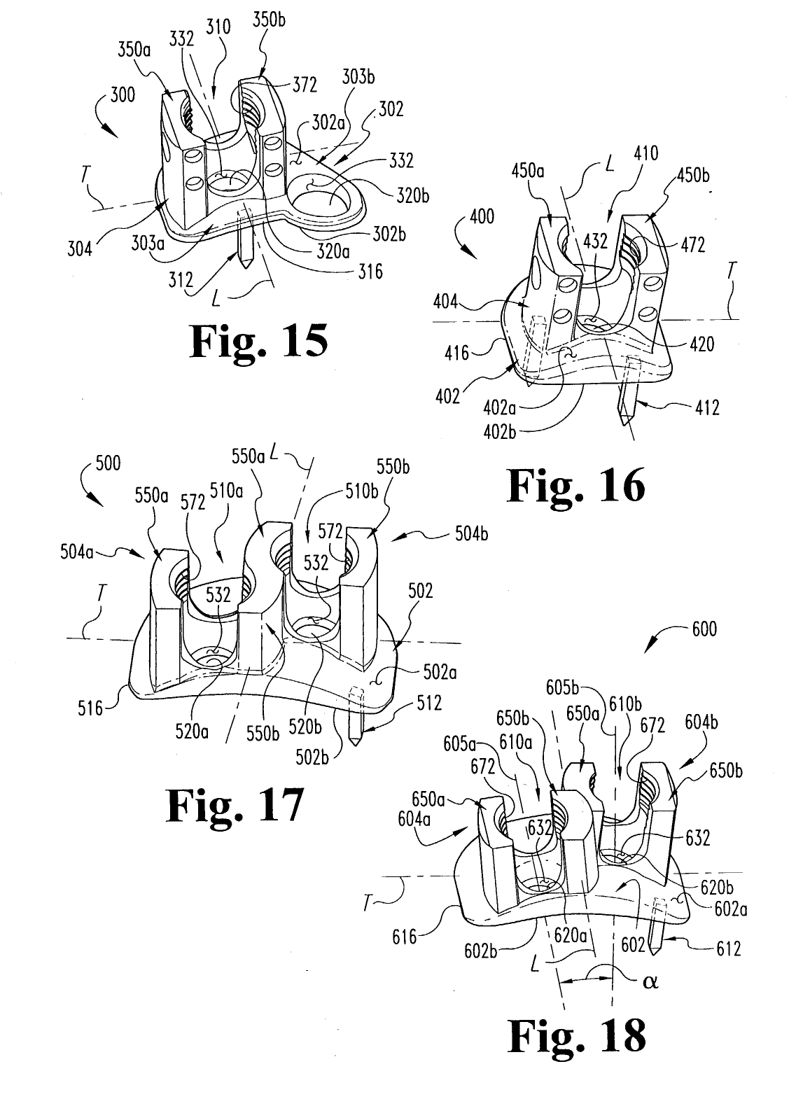

Referring to FIG. 15, shown therein is a spinal plate 300 according to yet another form of the present invention. The spinal plate 300 is configured similar to the spinal plate 100 illustrated and described above, including a plate portion 302 and a connector portion 304 integrally attached to the plate portion 302 and defining a channel 310 sized and shaped to receive an elongate rod therein. However, the plate portion 302 has a smaller axial and lateral profile in comparison to the plate portion 102 of the spinal plate 100. Additionally, unlike the rectangular configuration of the plate portion 102, the plate portion 302 has an irregular shape, including a central base portion 303a to which the connector portion 304 is attached, and a side or wing portion 303b projecting from the base portion 303a. At least one bone engagement member extends from the plate portion 302 for engagement with bone.

The plate portion 302 has a first side 302a facing away from the vertebral body and an opposite second side 302b facing the vertebral body. The connector portion 304 extends from and is integrally attached to the upper side 302a of the base portion 303a of the plate portion 302. The lower side 302b of the plate portion 302 may define a contour or curvature that approximates the anatomic curvature associated with the vertebral body to which the spinal plate 300 is engaged. In one embodiment of the invention, one or more prong members or spikes 312 extend from the lower side 302b of the plate portion 302 to engage the spinal plate 300 to bone. The prongs 312 are preferably formed integral with the plate portion 302; however, other means and methods for attaching the prongs 312 to the plate portion 302 are also contemplated. The prongs 312 preferably extend from the base portion 303a of the plate portion 302 and are preferably inwardly offset relative to an outer peripheral edge 316 extending about the plate portion 302. In one embodiment of the invention, the prongs 312 are arranged generally along the longitudinal axis L and are positioned in general alignment with the channel 310 in the connector portion 304. However, it should be understood that other positions and arrangements of the prongs 312 are also contemplated as falling within the scope of the present invention.

In the illustrated embodiment of the invention, the plate portion 302 defines a pair of bone anchor openings 320a, 320b extending from the upper side 302a to the lower side 302b. The bone anchor opening 320a extends through the base portion 303a of the plate portion 302 and is generally aligned with the channel 310 in the connector portion 304. The bone anchor opening 320b extends through the wing portion 303b of the plate portion 302. In the illustrated embodiment, the bone anchor openings 320a, 320b are axially offset from one another relative to the longitudinal axis L; however, in other embodiments of the invention, the bone anchor openings 320a, 320b may be aligned with one another generally along the transverse axis T. The bone anchor openings 320a, 320b are sized and shaped to receive respective bone anchors therethrough from the upper side 302a to the lower side 302b to engage the spinal plate 300 to bone. An arcuate-shaped recess 332 preferably surrounds each of the bone anchor openings 320a, 320b for receiving a spherical shaped portion of the bone anchor therein to allow the bone anchors to be oriented within a range of angles relative to the plate portion 302 and relative to one another.

The connector portion 304 is preferably formed integral with the plate portion 302 so as to define a unitary, single-piece structure. However, it should be understood that other means and methods for attaching the connector portion 304 to the plate portion 302 are also contemplated as falling within the scope of the present invention, such as, for example, by welding, bonding, fastening, or any other method of attachment known to those of skill in the art. In one embodiment, the connector portion 304 is oriented such that the channel 310 is generally aligned along the longitudinal axis L. However, other orientations of the connector portion 304 are also contemplated as falling with the scope of the present invention, including orientations where the channel 310 is aligned generally along the transverse axis T or along another axis oriented at an oblique angle relative to the longitudinal axis L or the transverse axis T.

The connector portion 304 is configured similar to the connector portion 104 associated with the spinal plate 100, including a pair of legs or posts 350a, 350b extending from the upper side 302a of the plate portion 302 and spaced apart to define the channel 310 therebetween. In one embodiment of the invention, the channel 310 is substantially U-shaped; however, other shapes and configurations of the channel 310 are also contemplated. The channel 310 is sized and shaped to receive an elongate rod R therein in a top or side loading manner or in an axial direction. Internal threads 372 are formed along at least a portion of the legs 350a, 350b for threadingly receiving a set screw 170 to capture or clamp the elongate rod R within the channel 310.

Referring to FIG. 16, shown therein is a spinal plate 400 according to yet another form of the present invention. The spinal plate 400 is configured similar to the spinal plate 300 illustrated and described above, including a plate portion 402 and a connector portion 404 integrally attached to the plate portion 402 and defining a channel 410 sized and shaped to receive an elongate rod therein. However, the plate portion 402 does not include a wing portion projecting laterally outward away from the connector portion 404. Instead, the plate portion 402 is comprised entirely of a central base portion having a lateral profile approximately equal to the lateral profile of the connector portion 404. As a result, the spinal plate 400 has a reduced footprint size relative to the other embodiments of spinal plates illustrated and described herein. At least one bone engagement member extends from the plate portion 402 for engagement with bone.

The plate portion 402 has a first side 402a facing away from the vertebral body and an opposite second side 402b facing the vertebral body. The connector portion 404 extends from and is integrally attached to the upper side 402a of the plate portion 402. The lower side 402b of the plate portion 402 may define a contour or curvature that approximates the anatomic curvature associated with the vertebral body to which the spinal plate 400 is engaged. In one embodiment of the invention, one or more prong members or spikes 412 extend from the lower side 402b of the plate portion 402 to engage the spinal plate 400 to bone. The prongs 412 are preferably formed integral with the plate portion 402; however, other means and methods for attaching the prongs 412 to the plate portion 402 are also contemplated. The prongs 412 are preferably inwardly offset relative to an outer peripheral edge 416 extending about the plate portion 402. In one embodiment of the invention, the prongs 412 are arranged diagonally opposite one another relative to the longitudinal axis L and the transverse axis T, positioned at opposite corners of the plate portion 402. However, it should be understood that other positions and arrangements of the prongs 412 are also contemplated as falling within the scope of the present invention.

In the illustrated embodiment of the invention, the plate portion 402 defines a single bone anchor opening 420 extending from the upper side 402a to the lower side 402b and generally aligned with the channel 410 in the connector portion 404. The bone anchor opening 420 is sized and shaped to receive a bone anchor therethrough from the upper side 402a to the lower side 402b to engage the spinal plate 400 to bone. An arcuate-shaped recess 432 preferably surrounds the bone anchor opening 420 for receiving a spherical shaped portion of the bone anchor therein to allow the bone anchor to be oriented within a range of angles relative to the plate portion 402.

The connector portion 404 is preferably formed integral with the plate portion 402 so as to define a unitary, single-piece structure. However, it should be understood that other means and methods for attaching the connector portion 404 to the plate portion 402 are also contemplated as falling within the scope of the present invention, such as, for example, by welding, bonding, fastening, or any other method of attachment known to those of skill in the art. In one embodiment, the connector portion 404 is oriented such that the channel 410 is generally aligned along the longitudinal axis L. However, other orientations of the connector portion 404 are also contemplated as falling with the scope of the present invention, including orientations where the channel 410 is aligned generally along the transverse axis T or along another axis oriented at an oblique angle relative to the longitudinal axis L or the transverse axis T. The connector portion 404 is configured similar to the connector portion 304 associated with the spinal plate 300, including a pair of legs or posts 450a, 450b extending from the upper side 402a of the plate portion 402 and spaced apart to define the channel 410 for receiving an elongate rod R. Internal threads 472 are formed along at least a portion of the legs 450a, 450b for threadingly receiving a set screw 170 to capture or clamp the elongate rod R within the channel 410.

Referring to FIG. 17, shown therein is a spinal plate 500 according to yet another form of the present invention. The spinal plate 500 has a similar configuration as the spinal plate 200 illustrated and described above, including a plate portion 502 and a pair of connector portions 504a, 504b integrally attached to the plate portion 502. The connector portions 504a, 504b define a pair of channels 510a, 510b, respectively, that are sized and shaped to receive a corresponding pair of elongate rods therein. At least one bone engagement member extends from the plate portion 502 for engagement with bone.

The plate portion 502 is configured substantially similar to the plate portion 202 of the spinal plate 200, having a substantially rectangular configuration defining a length extending along a longitudinal axis L and a width extending along a transverse axis T. The plate portion 502 has a first side 502a facing away from the vertebral body and an opposite second side 502b facing the vertebral body. The connector portions 504a, 504b extend from and are integrally attached to the upper side 502a of the plate portion 502. The lower side 502b of the plate portion 502 may define a contour or curvature that approximates the anatomic curvature associated with the vertebral body to which the spinal plate 500 is engaged.

In one embodiment of the invention, one or more prong members or spikes 512 extend from the lower side 502b of the plate portion 502 to engage the spinal plate 500 to bone. The prongs 512 are preferably formed integral with the plate portion 502; however, other means and methods for attaching the prongs 512 to the plate portion 502 are also contemplated. The prongs 512 are preferably inwardly offset relative to an outer peripheral edge 516 extending about the plate portion 502. In one embodiment of the invention, the spinal plate 500 includes a pair of prongs 512 arranged diagonally opposite one another relative to the longitudinal axis L and the transverse axis T, positioned at opposite corners of the plate portion 502. However, it should be understood that any number of prongs 512 may be used in association with the spinal plate 500 and that other positions and arrangements of the prongs 512 are also contemplated as falling within the scope of the present invention.

In the illustrated embodiment of the invention, the plate portion 502 defines a pair of bone anchor openings 520a, 520b extending from the upper side 502a to the lower side 502b. The bone anchor opening 520a extends through the plate portion 502 and is generally aligned with the channel 510a in the connector portion 504a, while the bone anchor opening 520b extends through the plate portion 502 and is generally aligned with the channel 510b in the connector portion 504b. In the illustrated embodiment, the bone anchor openings 520a, 520b are arranged diagonally opposite one another relative to the longitudinal axis L and the transverse axis T, positioned at opposite corners of the plate portion 502 and arranged diagonally opposite the prongs 512. However, it should be understood that other arrangements and positions of the bone anchor openings 520a, 520b are also contemplated as falling within the scope of the present invention. The bone anchor openings 520a, 520b are sized and shaped to receive respective bone anchors therethrough from the upper side 502a to the lower side 502b to engage the spinal plate 500 to bone. An arcuate-shaped recess 532 preferably surrounds each of the bone anchor openings 520a, 520b for receiving a spherical shaped portion of the bone anchor therein to allow the bone anchors to be oriented within a range of angles relative to the plate portion 502 and relative to one another.

The connector portions 504a, 504b are preferably formed integral with the plate portion 502 so as to define a unitary, single-piece structure. However, it should be understood that other means and methods for attaching the connector portions 504a, 504b to the plate portion 502 are also contemplated as falling within the scope of the present invention, such as, for example, by welding, bonding, fastening, or any other method of attachment known to those of skill in the art. The connector portions 504a, 504b define a pair of channels 510a, 510b, respectively, that are sized and shaped to receive a pair of elongate rods. In one embodiment of the invention, the connector portions 504a, 504b are oriented such that each of the channels 510a, 510b are generally aligned along the longitudinal axis L and arranged substantially parallel to one another. However, it should be understood that other orientations of the connector portions 504a, 504b are also contemplated as falling with the scope of the present invention, including orientations where the channels 510a, 510b are aligned generally along the transverse axis T or along another axis oriented at an oblique angle relative to the longitudinal axis L or the transverse axis T. It should also be understood that the connector portions 504a, 504b need not necessarily be aligned along a common axis, but may instead be arranged such that the channels 510a, 510b are aligned along axes that are angularly offset from one another.

The connector portions 504a, 504b are configured similar to the connector portions 204a, 204b associated with the spinal plate 200, each including a pair of legs or posts 550a, 550b extending from the upper side 502a of the plate portion 502 and spaced apart to define the channels 510a, 510b therebetween. In the illustrated embodiment, the leg 550a of the connector portion 504a and the leg 550b of the connector portion 504b form a continuous central leg or post extending generally along the longitudinal axis L. In one embodiment of the invention, the channels 510a, 510b are substantially U-shaped; however, other shapes and configurations of channels are also contemplated. The channels 510a, 510b may be sized and shaped to receive an elongate rod therein in a top or side loading manner or in an axial direction. Internal threads 572 are formed along at least a portion of the legs 550a, 550b for threadingly receiving a set screw 170 to capture or clamp an elongate rod within each of the channels 510a, 510b.

Referring to FIG. 18, shown therein is a spinal plate 600 according to yet another form of the present invention. The spinal plate 600 is configured similar to the spinal plate 500 illustrated and described above, including a plate portion 602 and a pair of connector portions 604a, 604b integrally attached to the plate portion 602. The connector portions 604a, 604b define a pair of channels 610a, 610b, respectively, 29, that are sized and shaped to receive a corresponding pair of elongate rods therein. At least one bone engagement member extends from the plate portion 602 for engagement with bone.

The plate portion 602 is configured substantially identical to the plate portion 502 of the spinal plate 500, having a first side 602a facing away from the vertebral body and an opposite second side 602b facing the vertebral body. The connector portions 604a, 604b extend from the upper side 602a of the plate portion 602. The lower side 602b of the plate portion 602 may define a contour or curvature that approximates the anatomic curvature associated with the vertebral body to which the spinal plate 600 is engaged. One or more prong members or spikes 612 extend from the lower side 602b of the plate portion 602 to engage the spinal plate 600 to bone. The plate portion 602 also defines a pair of bone anchor openings 620a, 620b extending from the upper side 602a to the lower side 602b. The bone anchor opening 620a extends through the plate portion 602 and is generally aligned with the channel 610a in the connector portion 604a, while the bone anchor opening 620b extends through the plate portion 602 and is generally aligned with the channel 610b in the connector portion 604b. The bone anchor openings 620a, 620b are sized and shaped to receive respective bone anchors therethrough from the upper side 602a to the lower side 602b to engage the spinal plate 600 to bone. An arcuate-shaped recess 632 preferably surrounds each of the bone anchor openings 620a, 620b for receiving a spherical shaped portion of the bone anchor therein to allow the bone anchors to be oriented within a range of angles relative to the plate portion 602 and relative to one another.

The connector portions 604a, 604b are preferably formed integral with the plate portion 602 so as to define a unitary, single-piece structure. However, it should be understood that other means and methods for attaching the connector portions 604a, 604b to the plate portion 602 are also contemplated as falling within the scope of the present invention, such as, for example, by welding, bonding, fastening, or any other method of attachment known to those of skill in the art. In one embodiment of the invention, the connector portions 604a, 604b are oriented such that each of the channels 610a, 610b are generally aligned along the longitudinal axis L and arranged substantially parallel to one another. However, it should be understood that other orientations of the connector portions 604a, 604b are also contemplated as falling with the scope of the present invention, including orientations where the channels 610a, 610b are aligned generally along the transverse axis T or along another axis oriented at an oblique angle relative to the longitudinal axis L or the transverse axis T. It should also be understood that the connector portions 604a, 604b need not necessarily be aligned along a common axis, but may instead be arranged such that the channels 610a, 610b are aligned along axes that are angularly offset from one another.

The connector portions 604a, 604b are configured similar to the connector portions 504a, 504b associated with the spinal plate 500, each including a pair of legs or posts 650a, 650b extending from the upper side 602a of the plate portion 602 and spaced apart to define the channels 610a, 610b therebetween. In one embodiment of the invention, the channels 610a, 610b are substantially U-shaped; however, other shapes and configurations of channels are also contemplated. The channels 610a, 610b may be sized and shaped to receive an elongate rod therein in a top or side loading manner or in an axial direction. Internal threads 672 are formed along at least a portion of the legs 650a, 650b for threadingly receiving a set screw 170 to capture or clamp an elongate rod within each of the channels 610a, 610b.

Unlike the connector portions 504a, 504b associated with the spinal plate 500, which extend from the plate portion 502 in a substantially parallel manner, the connector portions 604a, 604b extend from the plate portion 602 at different angles or trajectories. More specifically, the connector portions 604a, 604b extend along trajectory axes 605a, 605b, respectively, that are offset from one another by an angle α. In one embodiment of the invention, the angle α is selected to correspond with the desired amount of angular offset between the bone anchors that extend through the bone anchor openings 620a, 620b in the plate portion 602. As illustrated in FIG. 14 and described above with regard to the spinal plate 200, in one embodiment of the invention, the anterior bone screw 122a is substantially aligned with the coronal plane, while the posterior bone screw 122b is oriented approximately 10 degrees in an anterior direction relative to the coronal plane. Accordingly, the angle α between the trajectory axes 605a, 605b of the connector portions 604a, 604b would be correspondingly set at approximately 10 degrees to correspond with the selected degree of angular offset between the bone screws 122a, 122b. However, it should be understood that other angles α are also contemplated as falling within the scope of the present invention.

The spinal plates 100, 200, 300, 400, 500 and 600 illustrated and described above made be formed of any suitable medical grade material, such as, for example, titanium, stainless steel, plastic, or other biocompatible and/or resorbable materials. The elongate rods R, R₁ and R₂ may also be formed of any suitable medical grade material, and may have a rigid or semi-rigid configuration or possibly a flexible or semi-flexible configuration.

While the invention has been illustrated and described in detail in the drawings and foregoing description, the same is to be considered as illustrative and not restrictive in character, it being understood that only the preferred embodiments have been shown and described, and that all changes and modifications that come within the spirit of the invention are desired to be protected.

What is claimed is:

1. A spinal plate, comprising:
a plate portion having a first side and an opposite second side;
a connector portion integrally attached to said first side of said plate portion and defining a channel sized to receive an elongate rod therein; and
at least one bone engagement member extending from said second side of said plate portion and adapted for engagement with bone.

2. The spinal plate of claim 1, wherein said at least one bone engagement member comprises at least one prong formed integral with said plate portion.

3. The spinal plate of claim 2, wherein said at least one bone engagement member comprises a pair of said at least one prong formed integral with said plate portion.

4. The spinal plate of claim 3, wherein said connector portion is disposed intermediate said pair of prongs.

5. The spinal plate of claim 3, wherein said pair of prongs are diagonally offset relative to one another.

6. The spinal plate of claim 2, wherein said plate portion defines an outer peripheral edge extending about an interior region, said at least one prong extending from said interior region of said plate portion.

7. The spinal plate of claim 1, wherein said plate portion defines at least one opening extending between said first side and said second side; and
wherein said bone engagement member comprises a bone anchor having a bone engaging portion extending through said at least one opening in said plate portion from said first side to said second side for engagement with bone.

8. The spinal plate of claim 7, wherein said at least one opening is laterally offset from said connector portion.

9. The spinal plate of claim 7, wherein a first of said at least one opening is generally aligned with said connector portion.

10. The spinal plate of claim 9, wherein a second of said at least one opening is laterally offset from said connector portion.

11. The spinal plate of claim 7, wherein said plate portion defines first and second ones of said at least one opening, said first and second openings diagonally offset relative to a longitudinal axis extending along the elongate rod.

12. The spinal plate of claim 7, wherein said first side of said plate portion defines a spherical-shaped recess extending about said at least one opening, said bone anchor including a spherical-shaped portion adapted for engagement within said spherical shaped recess.

13. The spinal plate of claim 1, wherein said connector portion includes a pair of opposing legs extending from said first side of said plate portion and spaced apart to define said channel therebetween; and
further comprising a clamp member displaceable along said opposing legs and into engagement with the elongate rod to clamp the elongate rod within said channel.

14. The spinal plate of claim 13, wherein said clamp member comprises a set screw.

15. The spinal plate of claim 1, wherein said channel is substantially U-shaped.

16. The spinal plate of claim 1, wherein said connector portion is integrally formed with said plate portion to define a unitary, single-piece structure.

17. The spinal plate of claim 1, wherein said second side of said plate portion defines a lateral curvature corresponding to an outer profile of a vertebral body to which the spinal plate is engaged.

18. The spinal plate of claim 1, further comprising first and second ones of said connector portion integrally attached to said first side of said plate portion, said first and second connector portions defining first and second channels each sized to receive an elongate rod therein.

19. The spinal plate of claim 18, wherein said first and second connector portions are diagonally offset relative to one another.

20. A spinal plate, comprising:
a plate portion having a first side and an opposite second side;
a first connector portion integrally attached to said first side of said plate portion and defining a first channel sized to receive a first elongate rod therein; and
a second connector portion integrally attached to said first side of said plate portion and defining a second channel sized to receive a second elongate rod therein;
at least one bone engagement member extending from said second side of said plate portion and adapted for engagement with bone.

21. The spinal plate of claim 20, wherein said first and second connector portions are diagonally offset relative to one another.

22. The spinal plate of claim 20, wherein said first and second connector portions are integrally formed with said plate portion to define a unitary, single-piece structure.

23. The spinal plate of claim 20, wherein said plate portion defines at least one opening extending between said first side and said second side; and
wherein said bone engagement member comprises a bone anchor having a bone engaging portion extending through said at least one opening in said plate portion from said first side to said second side for engagement with bone.

24. The spinal plate of claim 23, wherein said at least one opening is laterally offset from each of said first and second connector portions.

25. The spinal plate of claim 23, wherein a first of said at least one opening is generally aligned with said first connector portion, and wherein a second of said at least one opening is generally aligned with said second connector portion.

26. The spinal plate of claim 25, wherein said first connector portion and said first opening extend along a first axis, said second connector portion and said second opening extend along a second axis angularly offset relative to said first axis.

27. The spinal plate of claim 23, wherein said plate portion defines first and second ones of said at least one opening, said first and second openings arranged diagonally opposite one another.

28. The spinal plate of claim 27, wherein said first opening is generally aligned with said first connector portion, and wherein said second opening is generally aligned with said second connector portion.

29. The spinal plate of claim 20, wherein said at least one bone engagement member comprises at least one prong extending from said second side of said plate portion and formed integral with said plate portion.

30. The spinal plate of claim 29, wherein said at least one bone engagement member comprises first and second ones of said at least one prong.

31. The spinal plate of claim 30, wherein said first prong extends generally opposite said first connector portion, said second prong extending generally opposite said second connector portion.

32. The spinal plate of claim 30, wherein said first and second prongs are diagonally offset relative to one another.

33. The spinal plate of claim 32, wherein said first and second connector portions are diagonally offset relative to one another, said first and second prongs arranged diagonally opposite said first and second connector portions.

34. The spinal plate of claim 30, wherein said plate portion defines an outer peripheral edge extending about an interior region, said first and second prongs extending from said interior region of said plate portion.

35. A spinal plate, comprising:
a plate portion having a first side and an opposite second side, said plate portion defining at least one opening extending between said first and second sides;
at least one connector portion formed integral with said plate portion, said connector portion extending from said first side of said plate portion and defining a channel sized to receive an elongate rod therein;
at least one prong formed integral with said plate portion, said at least one prong extending from said second side of said plate portion for engagement with bone; and
at least one bone anchor having a bone engaging portion extending through said at least one opening in said plate portion from said first side to said second side for engagement with bone.

36. The spinal plate of claim 35, wherein said at least one bone engagement member comprises a pair of said at least one prong formed integral with said plate portion, said pair of prongs diagonally offset relative to one another.

37. The spinal plate of claim 35, wherein said plate portion defines first and second ones of said at least one opening, said first and second openings diagonally offset relative to one another.

38. The spinal plate of claim 35, wherein said first side of said plate portion defines a spherical-shaped recess extending about said at least one opening, said bone anchor including a head portion having a spherical-shaped portion adapted for engagement within said spherical shaped recess.

39. The spinal plate of claim 35, wherein said connector portion includes a pair of opposing legs extending from said first side of said plate portion and spaced apart to define said channel therebetween; and
further comprising a clamp member displaceable along said opposing legs and into engagement with the elongate rod to clamp the elongate rod within said channel.

40. The spinal plate of claim 39, wherein said channel is substantially U-shaped.

41. The spinal plate of claim 35, wherein said second side of said plate portion defines a lateral curvature corresponding to an outer profile of a vertebral body to which the spinal plate is engaged.

42. The spinal plate of claim 35, further comprising first and second ones of said connector portion integrally attached to said first side of said plate portion, said first and second connector portions defining first and second channels each sized to receive an elongate rod therein.

43. The spinal plate of claim 42, wherein said first and second connector portions are diagonally offset relative to one another.

44. The spinal plate of claim 42, wherein a first of said at least one opening is generally aligned with said first connector portion, and wherein a second of said at least one opening is generally aligned with said second connector portion.

45. The spinal plate of claim 44, wherein said first connector portion and said first opening extend along a first axis, said second connector portion and said second opening extend along a second axis angularly offset relative to said first axis.

46. The spinal plate of claim 42, wherein said plate portion defines first and second ones of said at least one opening, said first and second openings arranged diagonally opposite said first and second connection portions.

47. The spinal plate of claim 42, comprising first and second ones of said at least one prong extending from said second side of said plate portion, said first prong arranged generally opposite said first connector portion, said second prong arranged generally opposite said second connector portion.

48. The spinal plate of claim 42, comprising first and second ones of said at least one prong extending from said second side of said plate portion, said first and second prongs arranged diagonally opposite said first and second connector portions.

49. The spinal plate of claim 42, wherein said first and second channels are arranged substantially parallel to one another so as to receive the elongate rods in a substantially parallel relationship.

50. A spinal plate, comprising:
a plate portion having a first side and an opposite second side;
means for connecting at least one elongate rod to said plate portion, said means for connecting extending from said first side of said plate portion and formed integral therewith; and
means for engaging said plate portion to bone, said means for engaging extending from said second side of said plate portion.
